(19) United States
(12) Patent Application Publication
(10) Pub. No.: US 2025/0186245 A1
(43) Pub. Date: Jun. 12, 2025

(54) STANDING URINATION DEVICE

(71) Applicant: humangear, Inc., San Francisco, CA (US)

(72) Inventors: Christopher A. MIKSOVSKY, San Francisco, CA (US); Katrin M. WARD, San Francisco, CA (US)

(21) Appl. No.: 18/772,921

(22) Filed: Jul. 15, 2024

Related U.S. Application Data
(60) Provisional application No. 63/608,432, filed on Dec. 11, 2023.

(51) Int. Cl. A61F 5/455; A61F 5/44

(52) U.S. Cl. CPC A61F 5/4556; A61F 5/4408

(57) ABSTRACT

A standing urination device that permits a human with a female urethra to direct their urine into a toilet or onto a surface such as the ground while in a standing position is disclosed. The device is formed of a sheet material and can be folded into an appreciably compact, flat form for transport and storage. The device may be reconfigured into a generally elongated lateral and downwards funnel-shaped configuration with a wide, generally vulval and curving rim opening and an exit spout some distance forwards of the user. Tabs and releasable fasteners are used to hold the device in position.

### CROSS-REFERENCE TO RELATED APPLICATIONS

This application claims priority of U.S. Provisional Application No. 63/608,432, filed on Dec. 11, 2023, which is incorporated herein by reference in its entirety.

### TECHNICAL FIELD

The present invention relates, in general, to a urination device and, more particularly, to a urination device which permits a human with a female urethra to urinate while in a standing position while directing the flow of urine away from the person's body and clothing. Note that this may apply to a woman, trans-man, non-binary person or any human with a female urethra. For brevity, the term “woman” (plural “women”, “she/her” pronouns) will be used in this application.

### BACKGROUND ART

A natural problem encountered by women is the need to urinate in a normal, toilet-sitting position in situations that are unsanitary, uncomfortable, or simply inconvenient. Public restrooms are used by innumerable people on a daily basis and as can be imagined, become breeding grounds for pathogens such as bacteria, viruses, and fungi. Poor maintenance of these restroom facilities results in unsightly, grimy, and foul conditions. Because of this situation, many women are required to place themselves in a sitting position “hovering” above the toilet seat when using such restrooms so as to avoid contact with a dirty or unsanitary toilet seat. This is a very uncomfortable and unpleasant position for a woman to be in when urinating. Similarly, when camping or traveling, a restroom may not be readily available necessitating that a woman assume a bent or squatting position when urinating while having moved her clothing out of the way. This requires the woman to push down any clothing covering her vaginal area, thereby exposing her crotch, rear buttocks, and upper legs, which can be uncomfortable in cold conditions and indiscrete if there are other people nearby. Furthermore, the squatting position can be difficult for some women, such as elderly or people with joint or mobility issues.

Numerous products are available to allow women to circumvent the conventional toilet sitting position while urinating. These products are primarily intended for women who wish to avoid using unsanitary public restrooms as well as women who do not have direct access to restrooms, for example in the outdoors. Female urination devices (FUDs), also known as stand-to-pec (STP) devices, generally encompass devices featuring an open entryway connected to a cone shaped funnel or elongated nozzle to direct the flow of urine away from the user's body.

The designs commonly found on many STP devices, typically made of rigid or semi-rigid plastics and elastomers, contribute to an overall increase in size and weight of the devices while hindering their collapsibility and portability. Another common deficiency found in the prior art is the lack of a properly conformed seal between a device and the user's body. This often results in leakage onto the user's hands and/or clothes as urine enters into the device. An additional problem exhibited by some devices, particularly those made of softer, elastomeric materials, is that these devices do not have sufficient structural integrity—this lack of stiffness makes them unable to assist in holding clothing down and away from the user's crotch area during use.

### SUMMARY OF THE INVENTION

The present invention relates to standing urination devices that allows women to urinate in a standing position while directing the flow of urine away from their bodies. In some embodiments a portable urination device comprises a fluid collecting body with an appreciably triangular-shaped interior surface and a triangular exterior surface. The fluid collecting body has a generally vulval opening that is delineated by a rim that is shaped to receive the bilateral symmetry of a female human crotch. The interior surface delineates a fluid collecting portion that is positioned above a spout, wherein the spout is directed towards the front end of the fluid collecting body. During urination, urine is able to enter the fluid collecting portion of the fluid collecting body through the vulval opening before traveling downward both by the force of gravity and by the force of the urine itself being expelled from the body and exiting through the spout. The material of the present invention is somewhat pliable in order to allow the user to firmly press the device against their body to prevent leakage.

An object is to allow a woman to urinate in a standing position. To use the standing urination device, a woman positions the fluid collecting body such that the rim is aligned with her labia majora and urinates into the interior of the fluid collecting body. The angular interior surface prevents the spraying urine from exiting the fluid collecting body during use, except through the spout. Urine enters the fluid collecting portion and exits the fluid collecting body through the spout and away from the user. Additionally, the present invention capitalizes on the force of gravity as urine is directed into the fluid collecting portion and forced out of the spout.

In some embodiments, the device is collapsible for improved portability due to the flexible material of the device. Fold lines or crease lines are positioned in the flat sheet material that allow various panels to be bent into certain positions that can combine to configure the device in the uscable funnel shape. Tabs and releasable fasteners (such as snaps) may be employed to hold the device in the funnel shape for use. The device may be formed into a relatively compact, folded-form for storage and transport. When the user desires to use the device, the flattened form can be unfolded as shown in the attached drawings and fastened into a final downwards-lateral funnel shape. After use, the device can be rinsed and then re-folded back into a compact, flat form for storage and transport.

In another aspect, a urination device is described that permits a woman to direct her urine into a toilet or onto the ground while in a standing position. The device is formed of a flat, sheet material, preferably a pliable plastic such as polypropylene sheet, although other plastics or papers, or plastic-coated-papers may also be used. The flat form features a variety of weakened folding lines that allow the device to be folded and formed into a compact inwards-folded transport form, and then into a outwards-folded use form. The use form creates a generally downwards and lateral funnel-shaped form. The opening of the use form features a rim which delineates a generally vulval opening. Snaps and tabs permit the device to achieve an appreciably triangular configuration with has sufficient rigidity to be firmly held in place by the user as well as holding clothes that have been pushed down out of the way (particularly clothes with elastic materials that may be inclined to ride up the user's legs). The device can be fabricated from a single piece of sheet material making for efficient and cost-effective manufacture.

In another aspect, a portable urination device is described that is configured to be transformed between a storage state and a use state. The portable urination device comprising a sheet having a plurality of panels that are configured to be folded to form a funnel shape when the portable urination device is in the use state. The plurality of panels including a first side panel, a second side panel, a first top panel and a second top panel. A first fold line is positioned between the first side panel and the first top panel and a second fold line is between the second side panel and the second top panel. At least one releasable top panel fastener is provided to facilitate attaching the first and second top panels in an overlapping manner when the portable urination device is in the use state with the sheet in the funnel shape.

In some embodiments, a third fold line is provided between the first and second side panels so that the portable urination device has a substantially triangular lateral cross section when in the use state.

In some embodiments, the device has a spout region that forms a spout and a heel region that forms a heel when the portable urination device is in the use state. The spout region and heel region are located on longitudinally opposite ends of the portable urination device when the portable urination device is in the use state. The sheet further comprises a lateral fold line that extends laterally across the first top panel, the first side panel, the second side panel and the second top panel. The lateral fold line facilitates folding the sheet such that a spout region resides near an interior of the heel region when the portable urination device is in the storage state. In some embodiments, the device can be transformed from the use state to the storage stat by: releasing the top panel fastener(s); folding the sheet along the lateral fold line such that that a spout region resides near an interior of the heel region; making a second fold along the longitudinal fold line; and thereafter securing a selected one of the top panel fasteners to hold the folded sheet in the storage position. In some embodiments, the selected fastener is a button top on the second top panel configured to snap into a button base on the first top panel in the use position and to snap into an opening on the first top panel in the storage position.

In some embodiments, the portable urination device further includes at least one tab on the first top panel and at least one slot in the second fold line. Each tab on the first top panel is configured to be inserted into a corresponding one of the slots when the portable urination device is in the use state with the sheet in the funnel shape. In other embodiments, the slots may be a bit below the second fold line. In some embodiments, there are two tabs and two slots.

In some embodiments, two releasable top panel fasteners are provided. In some embodiments, used to attach the first and second top panels in the overlapping manner. In some embodiments, the releasable top panels fasteners are snaps.

In some embodiments, a heel region of the sheet at a first end of the sheet includes a plurality of heel panels with heel fold lines between adjacent heel panels. The heel region configured to be folded to help form a heel of the portable urination device when the device is in the use state. In some embodiments, first and second heel fastener components are provided that together constitute a heel fastener. The sheet has a longitudinal centerline and the heel region includes first, second and third ones of the heel panels located on one side of the longitudinal centerline and a fourth one of the heel panels located on an other side of the longitudinal centerline. The first heel fastener component is located on the one side of the longitudinal centerline and the second heel fastener component is located on the fourth heel panel. The first, second and third heel panels are folded accordion style and the first second and third heel panels each have a hole therein through which the first heel fastener component engages the second fastener component when the portable urination device is in the use state.

In some embodiments, the sheet can be laid flat for storage with the one top panel fastener(s) disengaged. In some embodiments, the sheet has a single layer when laid flat. In some embodiments, the sheet can be folded into a compact stacked form for storage. In some embodiments, the sheet can be folded into a compact stacked form for storage with the heel fastener engaged.

In some embodiments, the sheet is formed from a plastic material or silicone. In some embodiments, the sheet is formed from polypropylene.

In some embodiments, the first and second top panels each have an associated attachment hole. The attachment holes are aligned when the portable urination device is in the use state with the sheet in the funnel shape.

In some embodiments, the attachment holes are sized suitably for receiving a carabiner, tether or other clip means for carrying the portable urination device when the portable urination device is in the use state, with each attachment hole being located near a distal end of the associated top panel.

In another aspect, a portable urination device is configured to be transformed between a storage state and a use state. The portable urination device includes a sheet having a plurality of panels and is configured to be folded to form a funnel shape when the device is in the use state. The panels include a first side panel, a second side panel, a first top panel and a second top panel. A first fold line is positioned between the first side panel and the first top panel and a second fold line is positioned between the second side panel and the second top panel. The sheet further includes at least one tab on the first top panel and at least one slot in or just below the second fold line. Each tab on the first top panel is configured to be inserted into a corresponding one of the slots when the device is in the use state.

In some embodiments there are two tabs and two slots.

In some embodiments, the device further includes a pair of releasable top panel fasteners that facilitate attaching the first and second top panels in an overlapping manner when the device is in the use state.

In some embodiments, first and second heel fastener components are provided that together constitute a heel fastener. The device includes a heel region having a plurality of heel panels with fold lines between adjacent heel panels. The heel region is configured to be folded to help form a heel of the device. The sheet has a centerline and the heel region includes first, second and third ones of the heel panels located on one side of the centerline and a fourth one of the heel panels located on an other side of the centerline. The first heel fastener component is located on the one side of the centerline. The second heel fastener component is located on the fourth heel panel. The first, second and third heel panels are folded accordion style when the device is in the use state. The first, second and third heel panels each have a hole therein through which the first heel fastener component engages the second fastener component when the device is in the funnel shaped use state.

In some embodiments, the sheet can be laid flat for storage and/or can be folded into a compact stacked form with each tab disengaged from its corresponding slot.

In another aspect a portable urination device is configured to be transformed between a storage state and a use state. The device includes a sheet having a plurality of panels configured to be folded to form a funnel shape when the device is in the use state. The panels include a first side panel, a second side panel, at least one top panel, a centerline, and a heal region at a first end of the sheet. The heal region has a plurality of heel panels with heel fold lines between adjacent heel panels. The heel region configured to be folded along the heel fold lines to help form a heel of the device when the portable urination device is in the use state. A heel fastener includes first and second heel fastener components. The heel region includes first, second and third heel panels located on one side of the centerline and a fourth heel panel located on the other side of the centerline. The first heel fastener component is located on the one side of the centerline and the second heel fastener component is located on the fourth heel panel. The first, second and third heel panels are folded accordion style when the portable urination device is in the use state. The first, second and third heel panels each have a hole therein through which the first heel fastener component engages the second fastener component when the portable urination device is in the use state.

In some embodiments, the heel fastener is a snap. In some embodiments, a fold line adjacent the fourth heel panel serves as a heel centerline in the use state. In some embodiments, the sheet can be laid flat for storage with the heel fastener disengaged, and the sheet can be folded into a compact stacked form for storage with the heel fastener engaged.

In another aspect a portable urination device is provided that is configured to be transformed between a planar storage state and a funnel shaped use state. The portable urination device is formed from a sheet having a plurality of panels and configured to be folded to form the funnel shape when the device is in the use state. The sheet can be unfolded to a substantially flat single layer.

In another aspect, a portable urination device is provided that is configured to be transformed between a storage state and a use state. The portable urination device includes a sheet having a plurality of panels and configured to be folded to form a funnel shape when the device is in the use state. The plurality of panels including a first side panel, a second side panel, a first top panel and a second top panel. The first and second top panels overlap when the device is in the use state. The first and second top panels each have an associated attachment hole located near a distal end of thereof. The attachment holes are aligned when the device is in the use state. The attachment holes are sized suitably for receiving a carabiner, tether or other clip means for carrying the portable urination device when the device is in the use state.

### BRIEF DESCRIPTION OF THE DRAWINGS

FIG. 1 is a perspective view of an embodiment of the present invention.

FIG. 2 is a top-down view of the embodiment of FIG. 1 in its flat form.

FIG. 3 is a bottom-up view of the embodiment of FIG. 1 in its flat form.

FIG. 4 is a detail perspective view of the heel area of the embodiment of FIG. 1 showing the Z-fold arrangement of the heel panels before the heel fasteners are attached.

FIG. 5 is a detail perspective view of the heel area of the embodiment of FIG. 1 showing the Z-fold arrangement of the heel panels after the heel fasteners are attached.

FIG. 6 is a top-down view of the embodiment of FIG. 1 with the heel portion in the fastened state.

FIG. 7 is a perspective view showing the spout of the device being inwards-folded into (or outwards-folded out of) the interior of the device, towards (or away from) the heel.

FIG. 8 is a top-down view showing the spout of the device after being inwards-folded into the interior of the device, towards the heel.

FIG. 9 is a top-down view showing the spout of the device after being inwards-folded into the interior of the device, towards the heel, with a fastener coupled to a hole to hold the device in a more compact shape.

FIG. 10 is a left side view showing the spout of the device after being inwards-folded into the interior of the device, towards the heel, with a fastener coupled to a hole to hold the device in a more compact shape.

FIG. 11 is a top-down view of the device with the heel portion in the fastened state showing the upper panels bent along fold lines towards each other.

FIG. 12 is a detail perspective view showing the arrangement of the top-lower-panel and top-upper-panel after tabs on the top-lower-panel have been inserted into mating slots on the top-upper-panel.

FIG. 13 is a detail perspective top-down view showing the arrangement of the top- lower-panel and top-upper-panel after tabs on the top-lower-panel have been inserted into mating slots on the top-upper-panel.

FIG. 14 is a detail perspective view looking from the heel of the device downwards towards the spout showing the arrangement of the top-lower-panel and top-upper-panel after tabs on the top-lower-panel have been inserted into mating slots on the top-upper-panel.

FIG. 15 is a detail perspective view looking from the heel of the device downwards towards the spout showing the arrangement of the top-lower-panel and top-upper-panel after tabs on the top-lower-panel have been inserted into mating slots on the top-upper-panel and after releasable fasteners on the upper panels have been coupled.

FIG. 16 is a top-down view showing the arrangement of the fully-assembled device. This shows the device with the top-lower-panel and top-upper-panel after tabs on the top-lower-panel have been inserted into mating slots on the top-upper-panel and after releasable fasteners on the upper panels have been coupled.

FIG. 17 is a perspective view showing the device being used.

FIG. 18 shows side views of the device being used at a toilet and in the outdoors.

FIG. 19 is a detailed perspective view showing a carabiner attached through holes near the spout of the device.

### DETAILED DESCRIPTION OF THE INVENTION

All illustrations of the drawings are for the purpose of describing selected versions of the present invention and are not intended to limit the scope of the present invention.

Referring next to the drawings, a portable standing urination device 10, also known as a stand-to-pee (STP) device or a female-urination-device (FUD), for directing the flow of urine away from a user's body will be described. The portable urination device comprises a fluid diverting body 20 that conveys urine from an opening defined by a rim 64 through a spout 60 some distance down and forwards from the user.

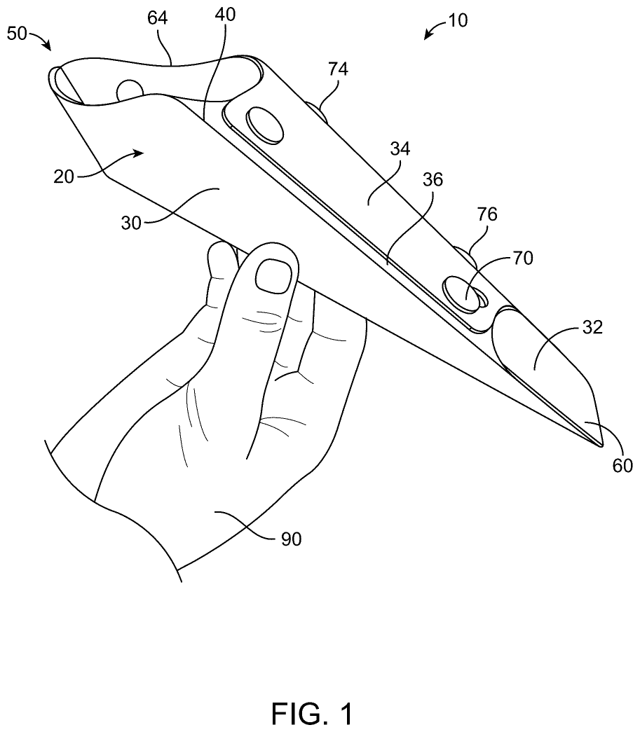

In reference to FIG. 1, the standing urination device 10 comprises a triangular-shaped fluid diverting body 20. The fluid diverting body 20 is defined by a left side panel 30, a right side panel 32, an top upper panel 34 and a top lower panel 36. When assembled as shown, the side panels and upper panels define a generally triangular cross-sectional shape. At one end towards the user the fluid diverting body features a curving rim 64 that is designed to encompass the vulvar region of a female human. Another end of the fluid diverting body 20 features a spout 60. A curving heel 50 is at the rearward end of the rim 64. Various fold lines 40 permit the panels to bend and fold along these lines, thereby permitting the device to be fashioned into its triangular use form from a flat sheet of material. Releasable fasteners such as snaps 70 may be used to secure the device into its use form. Tabs 74 are inserted into mating slot 76 to help align and fix the device into shape. The device 10 is held by a hand 90 during use.

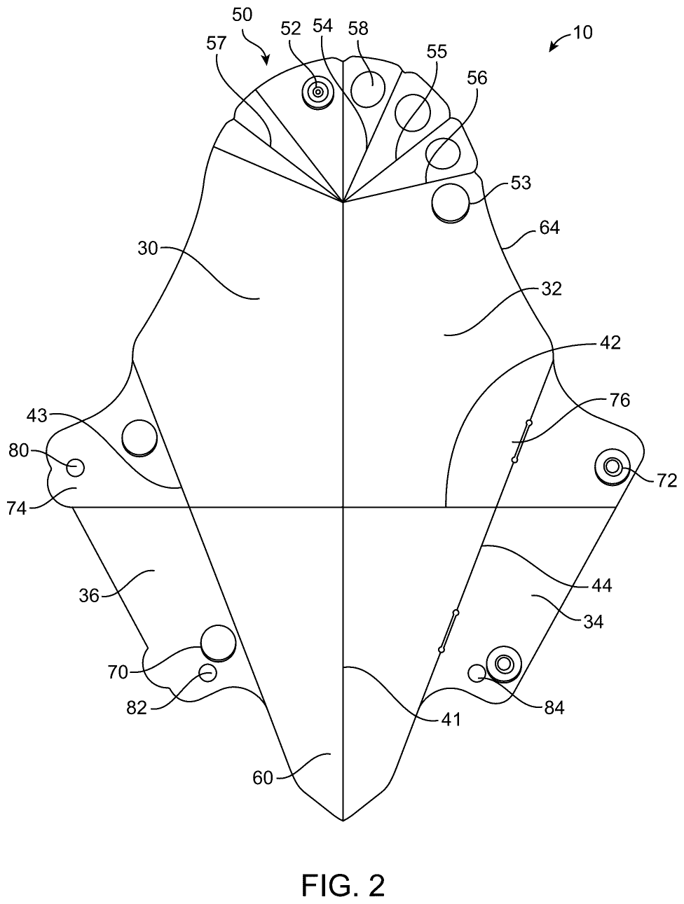

In reference to FIG. 2, the standing-urination-device 10 is shown in a flat form. The device 10 is preferably made from a sheet of plastic material such as polypropylene sheet, although other types of plastics, papers, and coated-papers may also be used. The device 10 is cut from the sheet material and weakened fold lines 40 are created to locally weaken the material and permit it to fold easily. These fold lines are also known as “living hinges”. In the preferred embodiment, the device 10 features a heel 50, a left side panel 30, a right side panel 32, a top upper panel 34, and a top lower panel 36.

In further reference to FIG. 2, the left side panel 30 and right-side panel 32 are defined by a center fold line 41. The top lower panel is defined by a left fold line 43. The top upper panel 34 is defined by a right fold line 44. The rim 64 of the assembled device is shown here in its flat form. One or more female snaps 70 may be coupled to one or more male snaps 72 to help secure the top lower panel to the top upper panel. One or more tabs 74 are insertable into one or more slots 76. Top lower panel hole 82 and top upper panel hole 84 are positioned such that they align when the device 10 is in an assembled state, thereby creating an attachment point for a clip or carabiner. A middle fold line 42 permits the end of the device with the spout 60 to fold inwards (or sideways) towards the device heel 50.

In further reference to FIG. 2, the heel 50 is defined by a curving edge and a multitude of fold lines such as a first Z-fold line 54, second Z-fold line 55, and a third Z-fold line 56, and a heel center line 57. A female fastener 52 is releasably coupled to a male fastener 53 to transform the heel area from a flat form into a three-dimensional curving form. Clearance holes 58 are used to permit the fasteners to couple with each other through the panels created by the various Z-fold lines (54, 55, 56). Note that the heel center line 57 is angled with respect to the center fold line 41. The angle of the Z-fold lines 54, 55, 56, are ⅓ the angle of the heel center line 57 relative to the center fold line 41. Furthermore, the heel center line 57 and the second Z-fold line 55, are symmetric about the center fold line 41.

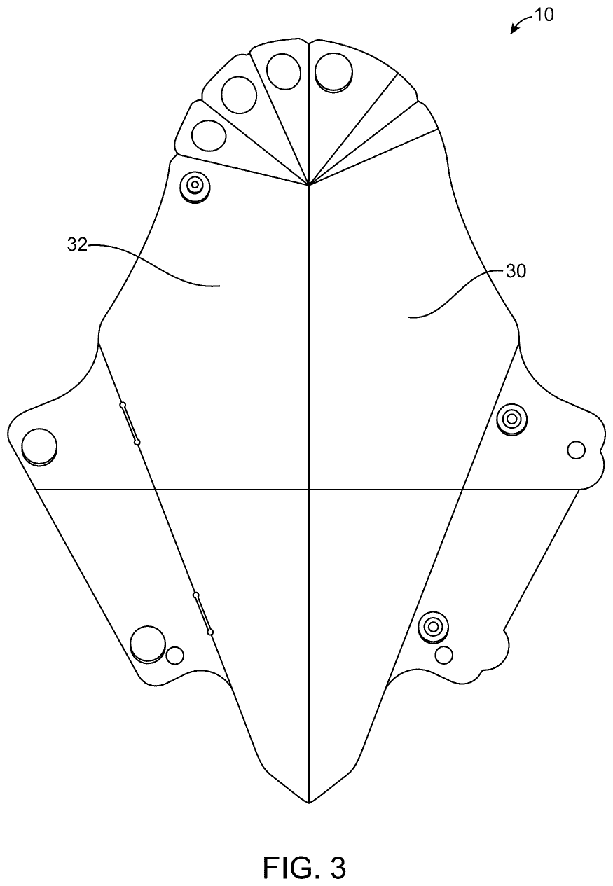

FIG. 3 shows the device 10 from the underside, showing the relative arrangement of the right-side panel 32 and left-side panel 30.

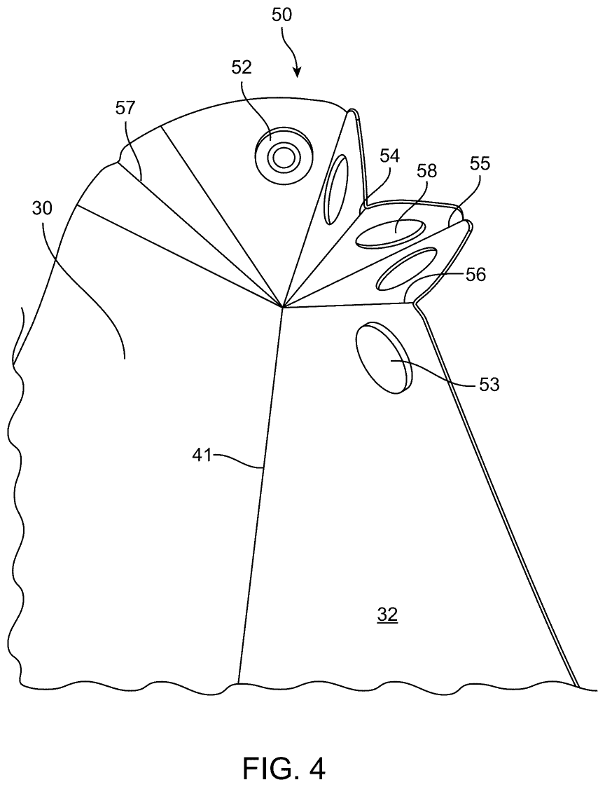

FIG. 4 shows the heel 50 of the device 10 during transformation from the manufactured flat form into the final, three-dimensional use form. The center fold line 41, first Z-fold line 54, second Z-fold line 55, and third Z-fold line 56 allow the heel panels to fold as shown in a zig-zag or Z-shaped configuration. A female fastener 52 and a male fastener 53 are positioned on the heel 50 and right-side panel 32, respectively. Fastener clearance holes 58 allow the female fastener 52 and the male fastener 53 to releasably couple each other through the folded panels of the heel 50. The left-side panel 30 is brought towards the right-side panel 32 as the heel Z-fold panels are brought together. Also note that with the zig-zag folding configuration, the heel center line 57 is brought into line with the center fold line 41.

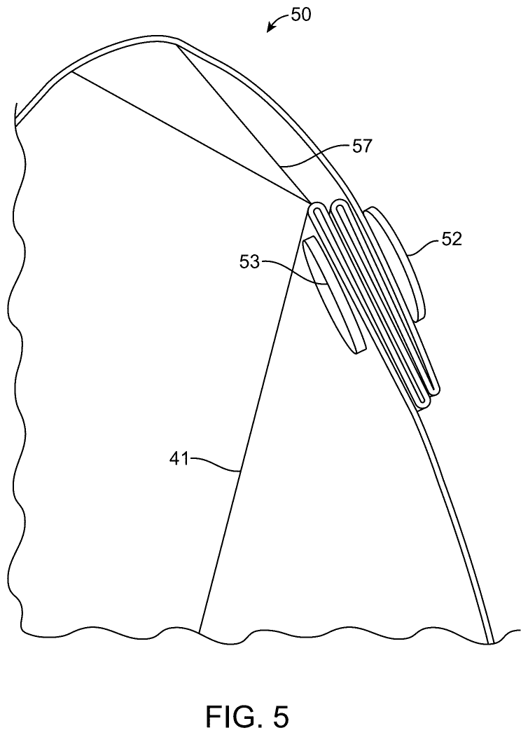

FIG. 5 shows the heel 50 of the device 10 after transformation from the manufactured flat form into the final, three-dimensional use form. The female fastener 52 has been releasably-coupled to the male fastener 53 to create the curving, three-dimensional heel form. The heel center line 57 is now fully in-line with the center fold line 41. This will permit the device to flatten along the center fold line 41, even with the heel still fully assembled and not in its prior flat state.

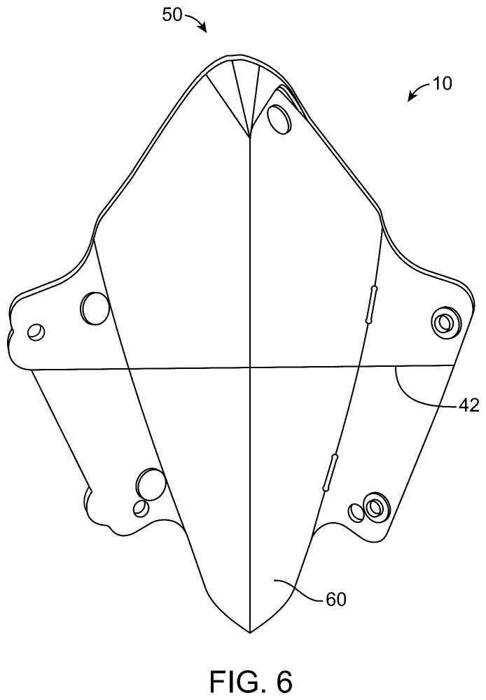

In reference to FIG. 6, the device 10 is shown with the heel 50 in its assembled form. A middle fold line 42 will permit the device to be folded approximately in half, with the spout 60 being folded inwards towards the heel 50.

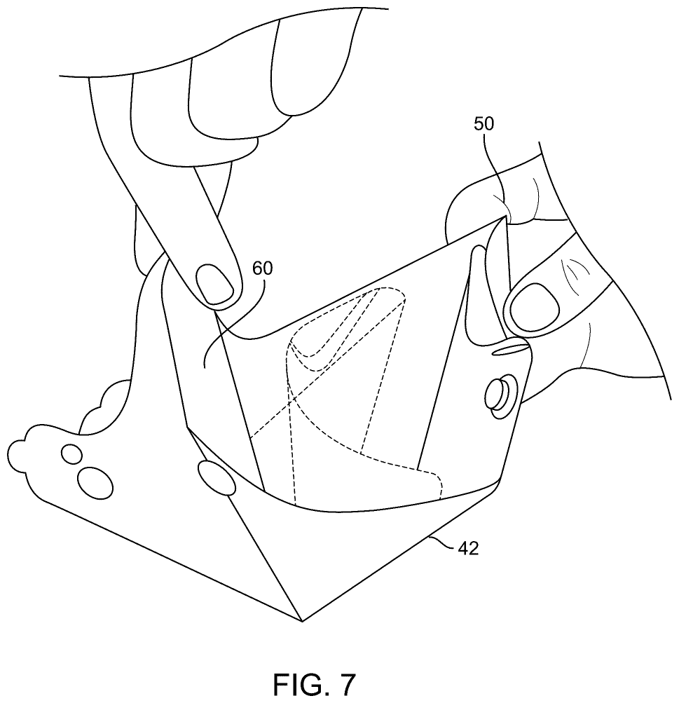

FIG. 7 shows one hand 90 of a user holding the assembled heel 50 of the device 10, with another hand 90 holding the spout 60. The spout 60 is pulled upwards and backwards, towards the heel 50, folding the device along the middle fold line 42. Dotted lines show a second position as the spout 60 is moved further towards the heel 50.

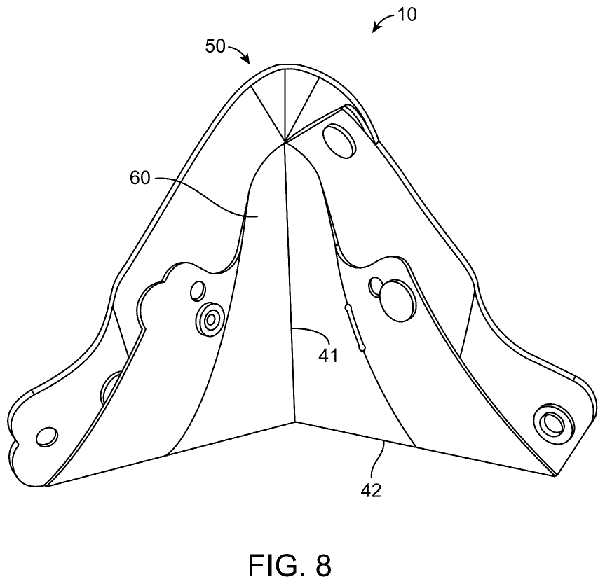

FIG. 8 shows the device 10 with the spout 60 fully folded inwards along the middle fold line 42. The spout 60 now resides near/in the interior of the heel 50. The center fold line 41 will now permit the folded left-and right-sides to come towards each other. Note that the heel center line 57 will also permit the left-and right-sides of the heel/device to come towards each other.

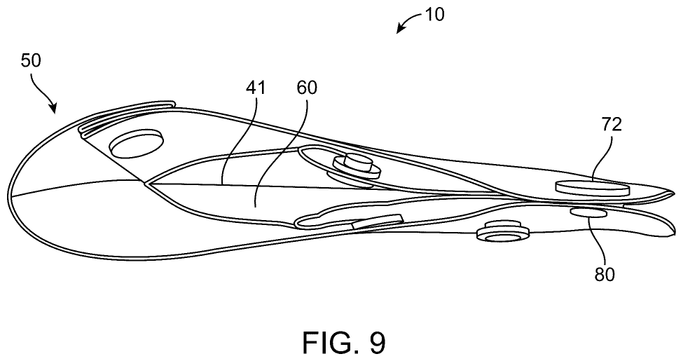

FIG. 9 is a top-down view showing the spout 60 of the device 10 folded inwards towards the heel 50. The left-and right-sides of the folded device can move towards each other along the center fold line 41. A male snap 72 is coupled to a hole 80 to hold the device in this folded arrangement. In other embodiments, a female snap component can be used in place of the hole 80.

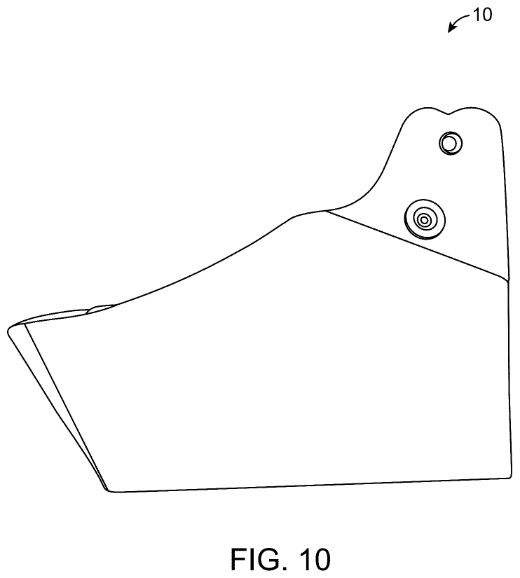

FIG. 10 is a left side view showing the side of the device in the folded form shown in FIG. 9.

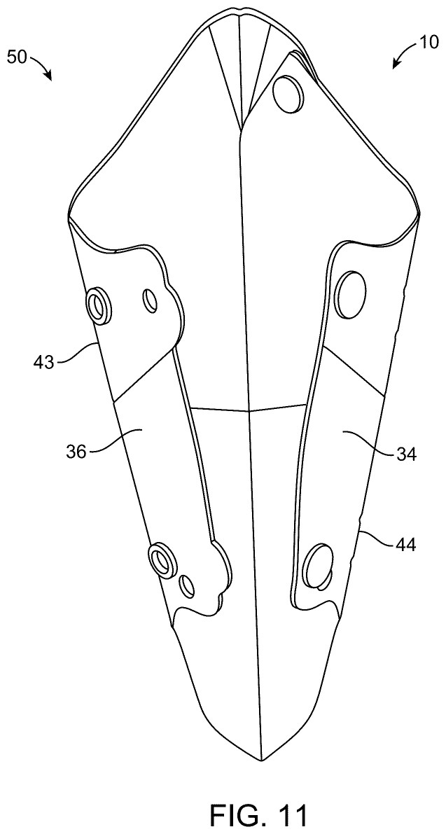

FIG. 11 is a top-down view showing the device 10 after the heel 50 has been assembled, with the top upper panel 34 and top lower panel 36 being folded towards each other along the left fold line 43 and right fold line 44.

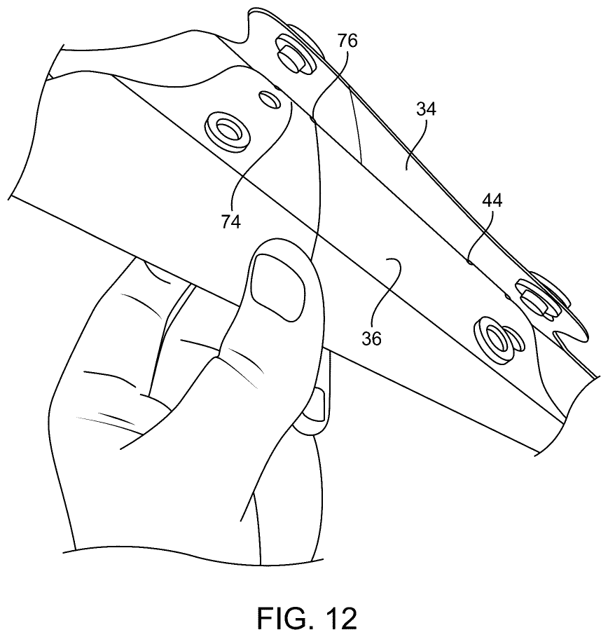

FIG. 12 is a detailed perspective view showing one or more tabs 74 of the top lower panel 36 being inserted into one or more mating slots 76 along the right fold line 44 that defines the top upper panel 34.

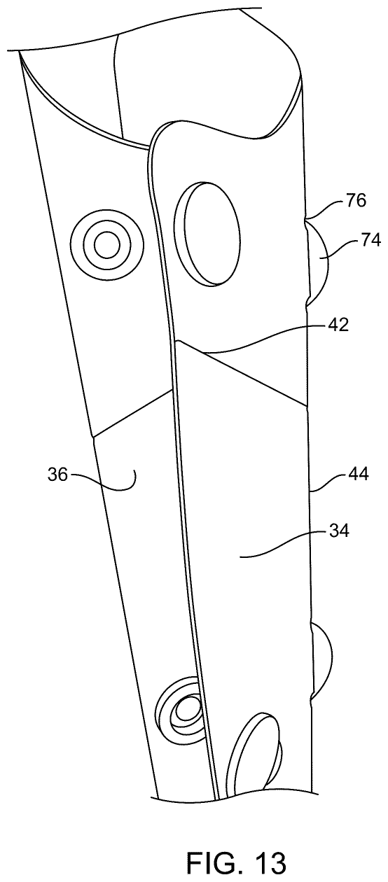

FIG. 13 is a top-down detail view showing one or more tabs 74 of the top lower panel 36 being inserted into one or more mating slots 76 along the right fold line 44 that defines the top upper panel 34. Note that here the middle fold line 42 is now split into sections (along the left and right sides of the assembled device) and as such the middle fold line no longer resides along one, straight line. As such, the device is not able to bend along the middle fold line 42 in this position.

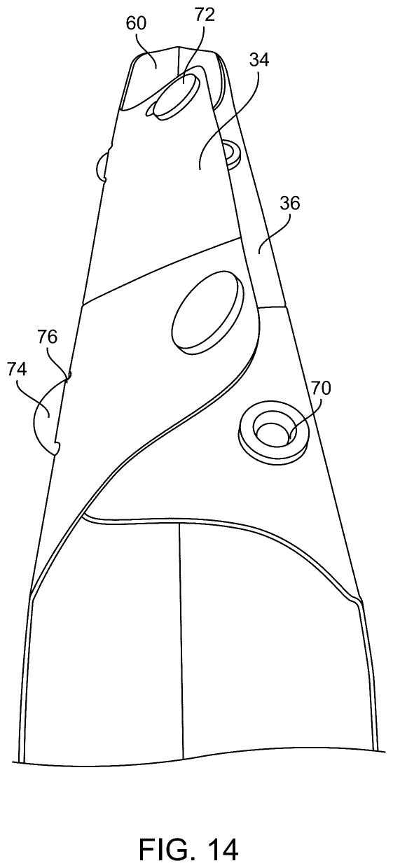

FIG. 14 is a perspective view towards the spout 60 of the device showing the relative arrangement of the top lower panel 36 and the top upper panel 34 prior to securing of one or more releasable couplers such as a female snap 70 and male snap 72. One or more tabs 74 are inserted into mating slots 76 to help position and secure the top lower panel 36 in place.

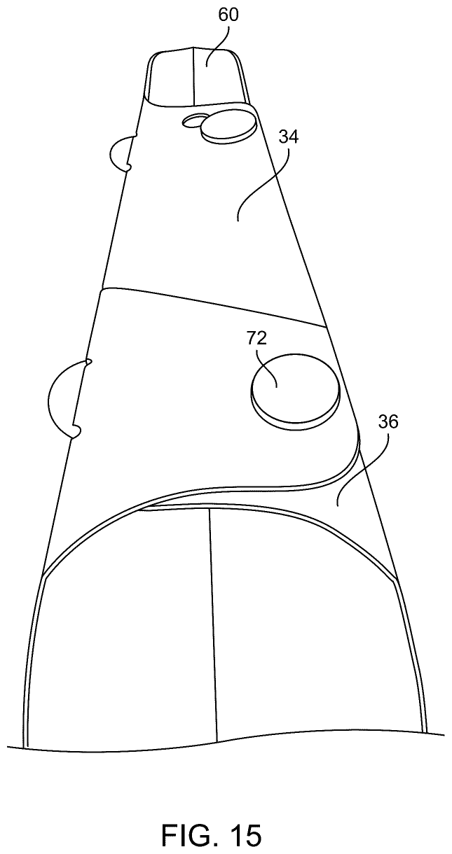

FIG. 15 is a perspective view towards the spout 60 of the device showing the relative arrangement of the top lower panel 36 and the top upper panel 34 after the securing of one or more releasable couplers such as male snap 72.

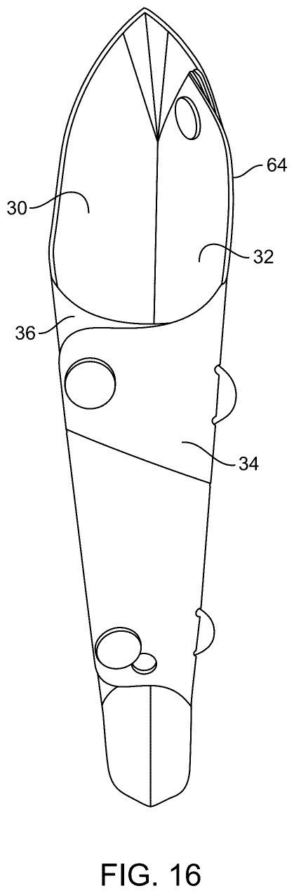

FIG. 16 is a top-down view towards of the fully-assembled device showing the curving rim 64 that this shaped to generally encompass the vulvar region of a female human. In this assembled state, the top upper panel 34 is laid on top of the top lower panel 36. These two panels combine to create the upper side of an appreciable triangular cross-sectional shape of the overall device 10. The left side panel 30 and right-side panel 32 define the other two sides of the triangular shape. This triangular shape is inherently rigid and stiff and helps the user hold items of clothing down and away from their body as they use the device.

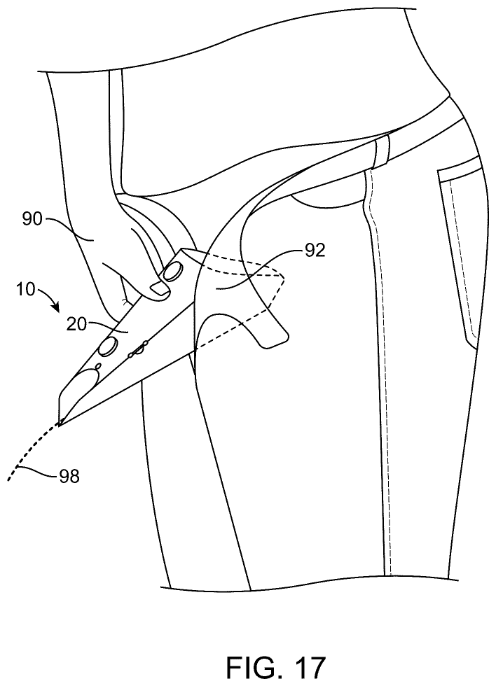

FIG. 17 is a perspective view showing the device 10 in use. A user uses their hand 90 to hold the device 10 upwards and against the vulvar region of their body. The triangular shape of the device 10 is relatively stiff and is able to hold clothing 92 down and away from the crotch area. Urine 98 is released from the urethra of the user and it flows down and away from the user's body along the downwards-funnel shape of the fluid diverting body 20. Note that the user is in a standing position.

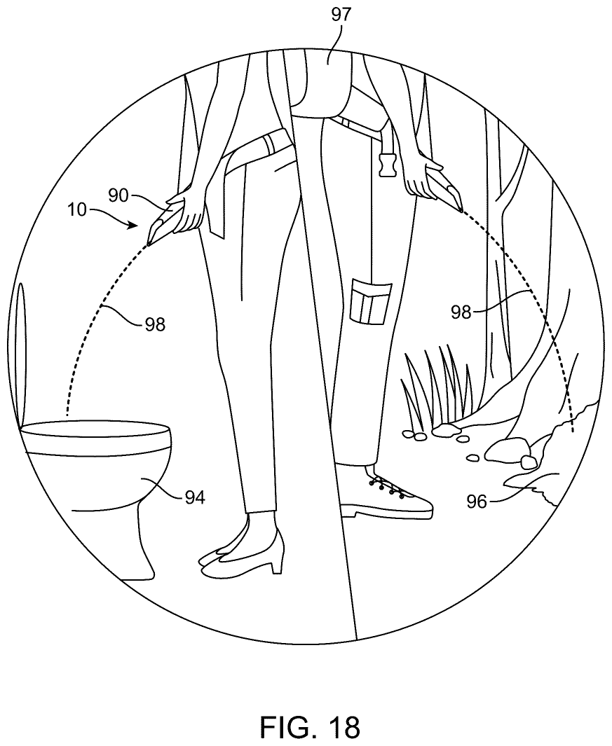

FIG. 18 shows two use scenarios of the device 10. On the left drawing, a user is holding the device 10 with their hand 90 and releasing their urine 98 through the device 10 into a toilet 94. Note that in this usage the user is able to use the toilet in a standing arrangement. The right-side drawing shows a similar use except in the outdoors, where the user can direct their urine 98 onto the ground or other outdoor surface 96. Note that in both use scenarios the user is able to keep their clothing 92 mostly on, which prevents them from having to expose their body to the elements or to the unseemly gaze of others that may be nearby. Also note that the user may keep other items such as a backpack 97 on their bodies during use, something they would typically need to take off were they to urinate in a typically seated or squatting position.

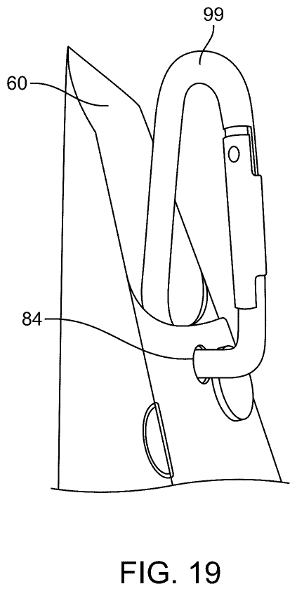

FIG. 19 is a detail perspective view showing the spout 60 end of the device 10 whereby the top lower panel hole 82 is aligned with the top upper panel hole 84. In this arrangement a small attachment device such as a carabiner 99 may be inserted through the holes 82 and 84 to thereby allow the device 10 to be attached to something else for transport or storage.

Certain modifications and improvements will occur to those skilled in the art upon reading the forgoing. It is understood that all such modifications and improvements have been deleted herein for the sake of conciseness and readability, but are properly within the scope this application.

What is claimed is:

1. A portable urination device configured to be transformed between a storage state and a use state, the portable urination device comprising:
a sheet having a plurality of panels and configured to be folded to form a funnel shape when the portable urination device is in the use state, the plurality of panels including a first side panel, a second side panel, a first top panel and a second top panel, there being a first fold line between the first side panel and the first top panel and a second fold line between the second side panel and the second top panel; and
at least one releasable top panel fastener configured to facilitate attaching the first and second top panels in an overlapping manner when the portable urination device is in the use state with the sheet in the funnel shape.

2. A portable urination device as recited in claim 1 further comprising a third fold line between the first and second side panels, whereby the portable urination device has a substantially triangular lateral cross section when in the use state.

3. A portable urination device as recited in claim 1 wherein:
the sheet has a spout region that forms a spout and a heel region that forms a heel when the portable urination device is in the use state, the spout region and heel region being on longitudinally opposite ends of the portable urination device when the portable urination device is in the use state; and
the sheet further comprises a lateral fold line that extends laterally across the first top panel, the first side panel, the second side panel and the second top panel to facilitate folding the sheet such that a spout region resides near an interior of the heel region when the portable urination device is in the storage state.

4. A portable urination device as recited in claim 1 wherein:
the sheet has a spout region that forms a spout when the portable urination device is in the use state and a heel region that forms a heel when the portable urination device is in the use state, the spout region and heel region being on longitudinally opposite ends of the portable urination device when the portable urination device is in the use state;
the sheet further comprises a longitudinal fold line and a lateral fold line, the longitudinal fold line extending between the first and second side panels and the lateral fold line extending laterally across the first top panel, the first side panel, the second side panel and the second top panel;
to transform the portable urination device from the use state to the storage state, the at least one top panel fastener is/are released, the sheet is folded along the lateral fold line such that that a spout region resides near an interior of the heel region, the sheet is additionally folded along the longitudinal fold line, and thereafter a selected one of the at least one releasable top panel fasteners is secured to hold the folded sheet in the storage position.

5. A portable urination device as recited in claim 4 wherein the selected fastener is a button top on the second top panel configured to snap into a button base on the first top panel in the use position and to snap into an opening on the first top panel in the storage position.

6. A portable urination device as recited in claim 1 further comprising at least one tab on the first top panel and at least one slot in the second fold line, wherein each tab on the first top panel is configured to be inserted into a corresponding one of the slots when the portable urination device is in the use state with the sheet in the funnel shape.

7. A portable urination device as recited in claim 6 wherein there are only two of the tabs and only two of the slots.

8. A portable urination device as recited in claim 1 wherein there are only two of the releasable top panel fasteners used to attach the first and second top panels in the overlapping manner.

9. A portable urination device as recited in claim 8 wherein the releasable top panels fasteners are snaps.

10. A portable urination device as recited in claim 1 wherein a heel region of the sheet at a first end of the sheet includes a plurality of heel panels with heel fold lines between adjacent heel panels, the heel region configured to be folded to help form a heel of the portable urination device when the portable urination device is in the use state with the sheet in the funnel shape.

11. A portable urination device as recited in claim 10 further comprising first and second heel fastener components that together constitute a heel fastener, and wherein:
the sheet has a longitudinal centerline and heel region includes first, second and third ones of the heel panels located on one side of the longitudinal centerline and a fourth one of the heel panels located on an other side of the longitudinal centerline;
the first heel fastener component is located on the one side of the longitudinal centerline;
the second heel fastener component is located on the fourth heel panel;
the first, second and third heel panels are folded accordion style when the portable urination device is in the use state with the sheet in the funnel shape; and
the first second and third heel panels each have a hole therein through which the first heel fastener component engages the second fastener component when the portable urination device is in the use state with the sheet in the funnel shape.

12. A portable urination device as recited in claim 11 wherein the first and second heel fastener components are components of a heel snap fastener.

13. A portable urination device as recited in claim 1 wherein the sheet can be laid flat for storage with each of the at least one top panel fasteners disengaged.

14. A portable urination device as recited in claim 1 wherein the sheet has a single layer when laid flat.

15. A portable urination device as recited in claim 1 wherein the sheet can be folded into a compact stacked form for storage with each of the at least one top panel fasteners disengaged.

16. A portable urination device as recited in claim 11 wherein the sheet can be folded into a compact stacked form for storage with the heel fastener engaged.

17. A portable urination device as recited in claim 1 wherein the sheet is formed from a plastic material or silicone.

18. A portable urination device as recited in claim 1 wherein the sheet is formed from polypropylene.

19. A portable urination device as recited in claim 1 wherein the first and second top panels each have an associated attachment hole, the attachment holes being aligned when the portable urination device is in the use state with the sheet in the funnel shape.

20. A portable urination device as recited in claim 19 wherein the attachment holes are sized suitably for receiving a carabiner, tether or other clip means for carrying the portable urination device when the portable urination device is in the use state with the sheet in the funnel shape, each attachment hole being located near a distal end of the associated top panel.

21. A portable urination device configured to be transformed between a storage state and a use state, the portable urination device comprising:
a sheet having a plurality of panels and configured to be folded to form a funnel shape when the portable urination device is in the use state, the plurality of panels including a first side panel, a second side panel, a first top panel and a second top panel, there being a first fold line between the first side panel and the first top panel and a second fold line between the second side panel and the second top panel;
at least one tab on the first top panel;
at least one slot in the second fold line; and
wherein each tab on the first top panel is configured to be inserted into a corresponding one of the slots when the portable urination device is in the use state with the sheet in the funnel shape.

22. A portable urination device as recited in claim 21 wherein there are only two of the tabs and only two of the slots.

23. A portable urination device as recited in claim 21 further comprising a pair of releasable top panel fasteners configured to facilitate attaching the first and second top panels in an overlapping manner when the portable urination device is in the use state with the sheet in the funnel shape.

24. A portable urination device as recited in claim 21 further comprising first and second heel fastener components that together constitute a heel fastener, and wherein:
a heel region of the sheet at a first end of the sheet includes a plurality of heel panels with fold lines between adjacent heel panels, the heel region is configured to be folded to help form a heel of the portable urination device when the portable urination device is in the use state with the sheet in the funnel shape;
the sheet has a centerline and heel region includes first, second and third ones of the heel panels located on one side of the centerline and a fourth one of the heel panels located on an other side of the centerline;
the first heel fastener component is located on the one side of the centerline;
the second heel fastener component is located on the fourth heel panel;
the first, second and third heel panels are folded accordion style when the portable urination device is in the use state with the sheet in the funnel shape; and
the first, second and third heel panels each have a hole therein through which the first heel fastener component engages the second fastener component when the portable urination device is in the use state with the sheet in the funnel shape.

25. A portable urination device as recited in claim 21 wherein:
the sheet can be laid flat for storage with each of the at least one tabs disengaged from the corresponding one of the one or more slots; and
the sheet can be folded into a compact stacked form for storage with each of the at least one tabs disengaged from the corresponding one of the one or more slots.

26. A portable urination device configured to be transformed between a storage state and a use state, the portable urination device comprising:
a sheet having a plurality of panels and configured to be folded to form a funnel shape when the portable urination device is in the use state, the plurality of panels including a first side panel, a second side panel, at least one top panel, a centerline, and a heal region at a first end of the sheet, the heal region having a plurality of heel panels with heel fold lines between adjacent heel panels, the heel region configured to be folded along the heel fold lines to help form a heel of the portable urination device when the portable urination device is in the use state with the sheet in the funnel shape; and
first and second heel fastener components that together constitute a heel fastener; and
wherein:
the heel region includes first, second and third ones of the heel panels located on one side of the centerline and a fourth one of the heel panels located on an other side of the centerline;
the first heel fastener component is located on the one side of the centerline;
the second heel fastener component is located on the fourth heel panel;
the first, second and third heel panels are folded accordion style when the portable urination device is in the use state with the sheet in the funnel shape; and
the first, second and third heel panels each have a hole therein through which the first heel fastener component engages the second fastener component when the portable urination device is in the use state with the sheet in the funnel shape.

27. A portable urination device as recited in claim 26 wherein the first and second heel fastener components are components of a heel snap fastener.

28. A portable urination device as recited in claim 26 wherein a fold line adjacent the fourth heel panel serves as a heel centerline in the use state.

29. A portable urination device as recited in claim 26 wherein:
the sheet can be laid flat for storage with the heel fastener disengaged; and
the sheet can be folded into a compact stacked form for storage with the heel fastener engaged.

30. A portable urination device configured to be transformed between a planar storage state and a funnel shaped use state, the portable urination device being formed from a sheet having a plurality of panels and configured to be folded to form the funnel shape when the portable urination device is in the use state and wherein the sheet can be unfolded to a substantially flat single layer.

31. A portable urination device configured to be transformed between a storage state and a use state, the portable urination device comprising:
a sheet having a plurality of panels and configured to be folded to form a funnel shape when the portable urination device is in the use state, the plurality of panels including a first side panel, a second side panel, a first top panel and a second top panel, the first and second top panels overlapping when the portable urination device is in the use state with the sheet in the funnel shape; and
wherein the first and second top panels each have an associated attachment hole located near a distal end of the associated top panel, the attachment holes being aligned when the portable urination device is in the use state with the sheet in the funnel shape; and
wherein the attachment holes are sized suitably for receiving a carabiner, tether or other clip means for carrying the portable urination device when the portable urination device is in the use state with the sheet in the funnel shape.
